(19) United States
(12) Patent Application Publication
(10) Pub. No.: US 2018/0311469 A1
(43) Pub. Date: Nov. 1, 2018

(54) ANTIMICROBIAL SHIELD AND BARRIER FOR URINARY CATHETER

(71) Applicant: Poiesis Medical, LLC, Jupiter, FL (US)

(72) Inventor: Gregory D. Wiita, Jupiter, FL (US)

(21) Appl. No.: 15/964,601

(22) Filed: Apr. 27, 2018

Related U.S. Application Data
(60) Provisional application No. 62/490,973, filed on Apr. 27, 2017.

(51) Int. Cl. A61M 25/01; A61M 25/00; A61L 29/16

(52) U.S. Cl. CPC A61M 25/0111; A61M 25/0017; A61L 2300/404; A61M 2025/0062; A61M 2025/0056; A61L 29/16

(57) ABSTRACT

An antimicrobial, antiseptic, and antibacterial barrier has a flexible shield configured to slidingly engage a urinary catheter. The shield has one or more sections configured to conform and adhere to the urinary meatus and/or surrounding skin or tissues of a target patient. A flexible expandable drape is engaged with the shield and is axially aligned with the catheter to provide an additional layer of protection to prevent the passage of bacteria, fungi, and contaminants into the urinary tract. The shield and drape each comprise an antimicrobial, antiseptic, and antibacterial material.

### CROSS REFERENCE TO RELATED APPLICATIONS

This application claims the benefit of U.S. Provisional Application Ser. No. 62/490,973 filed Apr. 27, 2017.

### BACKGROUND OF THE INVENTION

This invention is generally related to advanced antimicrobial, antiseptic, and antibacterial shields and physical barriers for use in conjunction with urinary catheters or other like ports, lines, and ducts inserted into the body.

Urinary catheters have been used for many years. A particularly well known urinary catheter is the Foley catheter. The Foley catheter includes a flexible tube made of latex and/or silicone material and has two internal lumens extending substantially parallel along the length of the tube. A balloon is positioned near the distal end of the tube for holding the catheter in position within the bladder of a patient. The catheter is positioned within the patient and fluid is introduced through one of the lumens to inflate the balloon to retain the catheter in the desired position. A drainage eye is located at the distal end of the catheter to allow the urine to pass through one of the lumens of the catheter tube for urine drainage purposes. However, the Foley catheter, like all other currently and commercially available catheters, suffers from the same or a similar drawback in that they have a tip that extends beyond the balloon. This tip often bears or scrapes against the liner of the bladder causing trauma to the bladder lining and is associated with numerous medical problems. A further problem relates to the position of the drain eye(s) causing patient discomfort when the mucosal lining is drawn into the drain eye(s). Some examples of the medical problems associated with the current catheter devices include discomfort, spasms and bleeding, as well as more serious drawbacks such as bacteria in the blood stream, e.g. urosepsis and urinary tract infections.

To solve the problems associated with traditional Foley catheters, Applicant developed the Duette catheter, a dual-balloon catheter described and claimed in U.S. Pat. No. 8,636,724. This novel catheter provides a catheter tip that includes a first balloon positioned to encapsulate the tip of the catheter and a second balloon to position the catheter within the urinary tract. The first balloon is constructed and arranged to cushion the catheter tip from traumatizing and irritating the internal wall of the cavity, duct or vessel. The first balloon is connected to the tip so as to prevent the tip from moving relative to the balloon. The second balloon, proximal from the first balloon functions to retain the catheter in the bladder. The first and second balloons are also effective in preventing mucosal tissue from being aspirated into the drainage eye of the catheter.

Although Applicant's dual balloon catheter has been empirically shown to reduce the occurrence of catheter-associated urinary tract infections (CAUTI), secondary sources of bacteria, fungi, and other microbial elements) entering the catheterization site (e.g. the area at and around the urethra and the spaces or gaps between the urinary meatus and the catheter) are a significant independent cause of urinary tract infections that are often mistakenly blamed on the catheter itself. Such secondary external sources of bacteria include bodily fluids (particularly feces) and unclean dermis in the areas surrounding the catheterization site. Importantly, an indwelling catheter system is often mischaracterized as a “closed system” which actually means only that the interior of the catheter and the barrier/seal at the drainage junctions are sterile, but the outside of the catheter system is not.

Significantly, because the catheter in both male and female patients resides in a bacteria and microbial-friendly environment, several studies demonstrate that bacteria outside of a “closed” catheter system are able to gain entry into the bladder at a more rapid pace then bacteria which may enter the bladder upon insertion of a catheter—this occurs because bacteria introduced during insertion is quickly flushed out of the urinary tract and into the collection system several feet away from the patient. Routine placement on indwelling catheters are mandated to be performed in an aseptic, but not necessarily sterile, manner, unlike surgical sites, peripherally inserted center catheters (PICC), intravenous lines, and wound sites, which are generally covered by sterile dressings to prevent bacteria from entering the site. Accordingly, there is a need in the art to physically shield and barrier the outside of the catheter system and reduce or eliminate the possibility of external bacteria at the catheterization site from entering the urinary tract. To be certain, while reduction of bladder damage reduces bacteria attachment sites and a significant reduction in aspiration blockage helps to more rapidly and completely clear any residual bacteria in the luminal space of the bladder, there is a need to prevent higher/extreme levels of bacteria from entering the lower urinary tract system from an external route i.e. outside of the catheter lumen.

### BRIEF DESCRIPTION OF THE DRAWINGS

The drawings appended hereto are mere schematics representations, not intended to portray specific parameters of the invention. Understanding that these drawing(s) depict only typical embodiments of the invention and are not, therefore, to be considered to be limiting its scope, the invention will be described and explained with additional specificity and detail through the use of the accompanying drawing(s), in which:

FIG. 1 is a schematic of one embodiment of the shield in a deployed position with an expanded drape.

FIG. 2 is a schematic of one embodiment of the shield in a retracted position with a retracted drape.

FIG. 3 is a perspective view of one embodiment of the shape of the shield.

FIG. 4 is a perspective view another embodiment of the shape of the shield.

FIG. 5 is a perspective view yet another embodiment of the shape of the shield.

FIG. 6 is a perspective view yet another embodiment of the shape of the shield.

FIG. 7A is a rear perspective view of yet another embodiment of the shield.

FIG. 7B is a front perspective view of the shield shown in FIG. 7A.

### DETAILED DESCRIPTION

For the purposes of promoting an understanding of the principles of this disclosure, reference will now be made to the exemplary embodiments illustrated in the drawing(s), and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to an “embodiment,” an “example” or similar language means that a particular feature, structure, characteristic, or combinations thereof described in connection with the embodiment is included in at least one embodiment of the present invention. Thus, appearances of the phrases an “embodiment,” an “example,” and similar language throughout this specification may, but do not necessarily, all refer to the same embodiment, to different embodiments, or to one or more of the figures. Additionally, reference to the wording “embodiment,” “example” or the like, for two or more features, elements, etc. does not mean that the features are necessarily related, dissimilar, the same, etc. The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional un-recited elements or method steps. As used herein the term “distal” generally is understood to mean that which is situated away from the center of the identified structure or from the point of attachment of said structure. The term “proximal” generally is understood to mean that which is situated nearer to the center of the body or to the point of attachment.

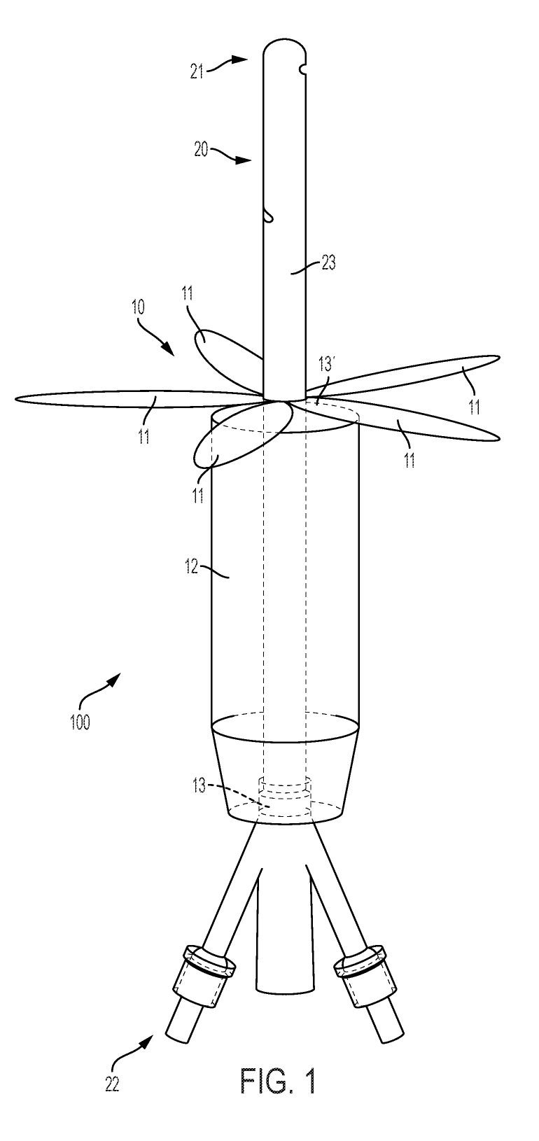

With reference to FIG. 1, shown is an embodiment of a barrier 100 comprising a shield 10 disposed on an exemplary catheter 20. The catheter comprises a distal end 21 configured to be inserted and drawn down into the bladder and a proximal end 22 providing the various drainage and insertion ports of the catheter 20. The shield 10 comprises a flexible physical barrier that may include one or more flexible sections or lobes 11 configured to resiliently adhere to, cover at least a portion of, and barrier the exposed urinary meatus (i.e. the opening of the urethra, situated on the glans penis in males, and in the vulva in females) of a catheterized human being or animal. In some embodiments, the lumen 23 is disposed through the shield 10, at opening 11′, such that the shield 10 is slidingly or expandably engaged with the lumen 23 so that following insertion of the catheter 20 the shield 10 can readily slide into position toward the distal end of the catheter to the urinary meatus. The sliding engagement of the shield 10 allows it to be adjustable to the patient's anatomy and other contributing factors when using a catheter 20.

In some embodiments, the barrier may also include a flexible, expandable drape 12 which functions as an additional barrier against bacteria during catheterization. In some embodiments, the drape 12 comprises a sheath-like member with a collapsible accordion or stepped, bellow-like configuration. In some embodiments, the drape 12 is attached or cohesive with the shield 10 at seal 13′ such that the barrier is slidingly or expendably engaged with the catheter 20. When the shield 10 is translated along the lumen 23 of the catheter 20, the drape 12 will expand and retract accordingly. In some embodiments the drape 12 includes a seal 13 adjacent to the proximal end 22 of the catheter 20. The seal 13 may comprise an adhesive or heat-sealed joint attaching at or about the proximal end of the drape 12 so as to close off the interior of the drape 12. A similar seal 13′ may, in some embodiments, be present at the junction of drape 12 and shield 10, providing the attachment thereof. This results in a “closed” system of protection around the catheterization site wherein medical personnel can access the drainage and insertion points without contaminating the covered area.

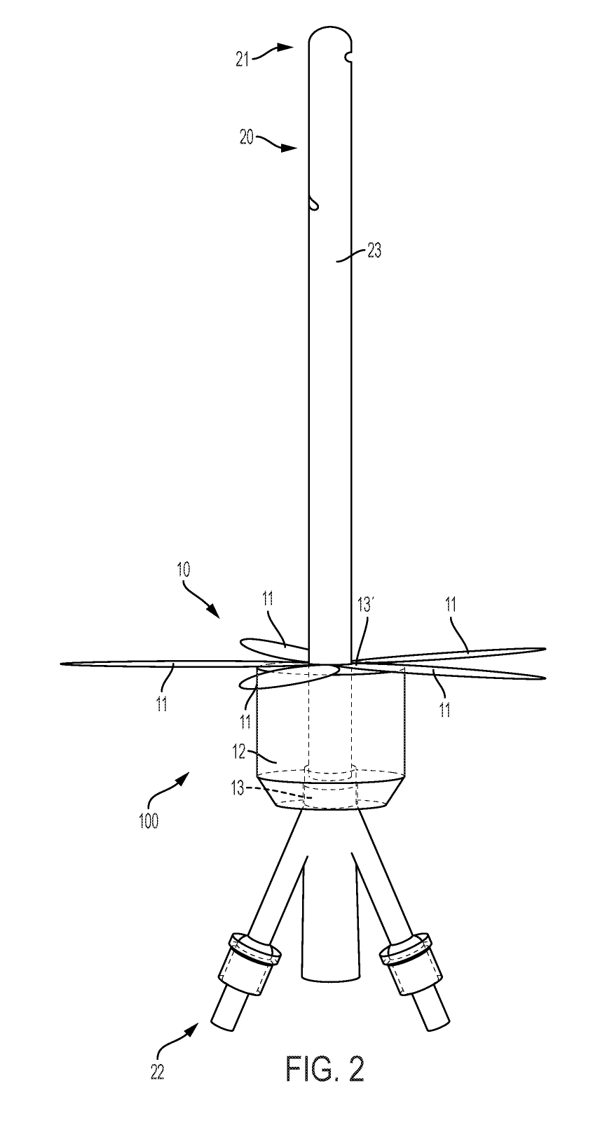

In a closed position, shown in FIG. 2, the drape 12 can be used to cover and hold the shield 10 during packaging, shipment, and before deployment and use. In an open or deployed position, the drape 12 is adjacent to and covers at least a portion of the catheterization site and surrounding area to provide an additional or supplementary barrier against bacteria. In some embodiments, the drape 12 is tubular and co-axially aligned and engaged with the lumen 23 of the catheter.

In some embodiments, the drape 12 is frangible and removable from the shield 10, for example, after the shield 10 is secured in position on the urinary meatus of the patient. The removed drape 12 can then be placed over the shield and general catheterization site for additional protection. The drape 12 functions to reinforce the barrier created by shield 10 and provide an initial and/or secondary physical barrier or layer of protection against the introduction of bacteria into the catheterization site. In some embodiments, the drape 12 is separate from the shield 10 such that it can be drawn down and around the catheterization site over and surrounding the shield 10. In yet other embodiments, the drape 12 is integrated into and deployable from a separate catheterization securement device attached to the patient near the catheterization site. These various embodiments of the drape 12 all provide an additional layer or barrier over the catheterization site to prevent the introduction of bacteria. Accordingly, the present invention contemplates a catheter protection system comprising a first barrier and a second barrier, whereby the first barrier (the drape 12) covers and protects the second barrier (the shield 10). The drape 12 can comprise a variety of shapes, sizes, and configurations to conform to the fitment and anatomy of a given patient.

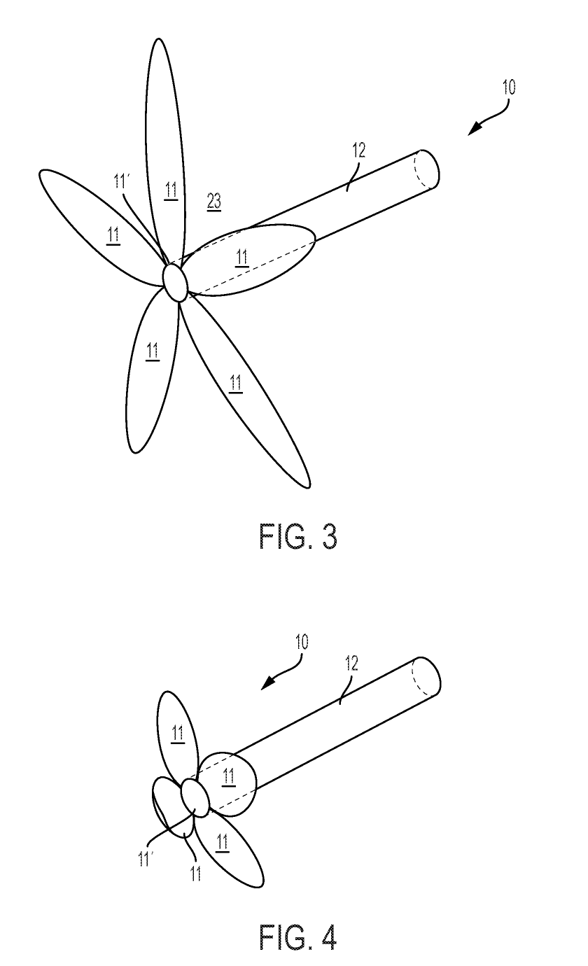

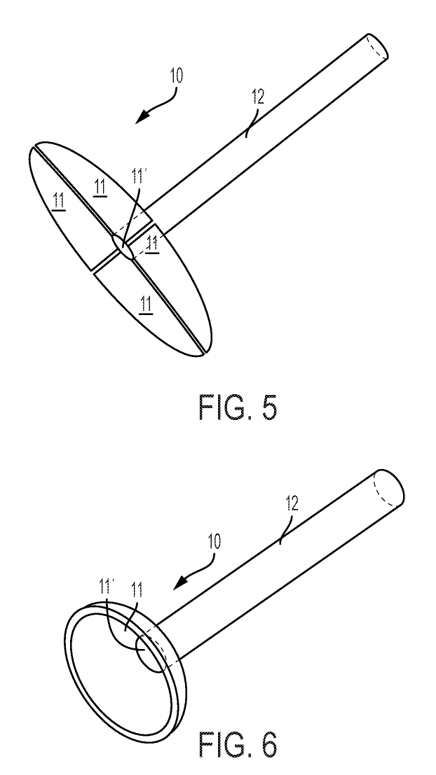

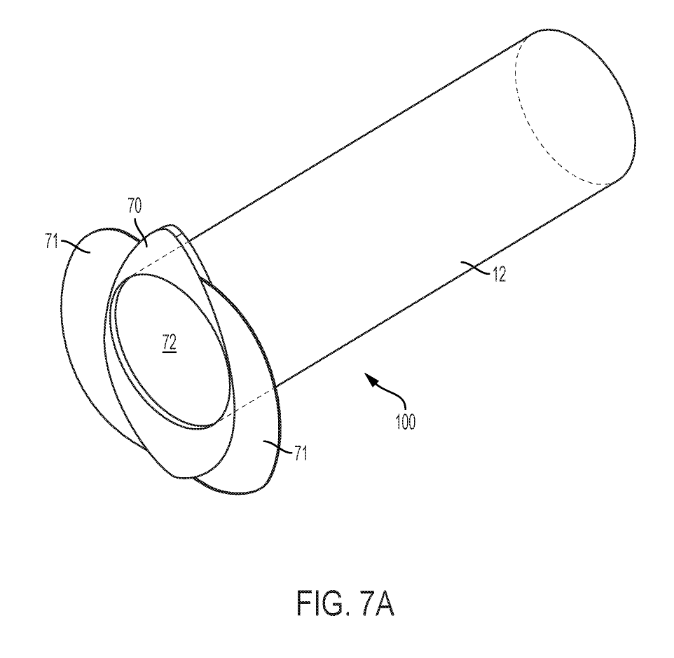

The size, shape, and configuration of the shield 10 can vary depending on the desired fitment and anatomy of a given patient and is configured to conform to such anatomy and form a protective barrier for both male and female patients. For example, in FIG. 3, the shield 10 comprises a “banana-peel” configuration having a plurality of elongated lobes 11. In FIG. 4, the shield comprises a “flower-petal” configuration with four orthogonal lobes 11. In FIG. 5, the shield comprises a section circular configuration, with four orthogonal lobes 11. In FIG. 6 the shield comprises a condom-like device with a circular cross-section configured to encase the urinary meatus and surrounding tissues (for example the glans).

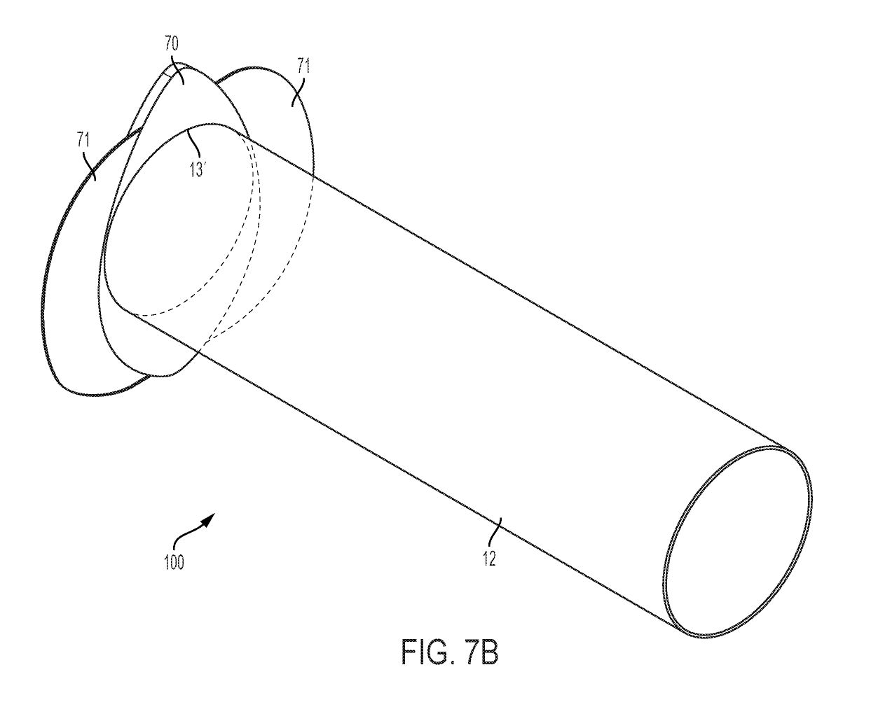

FIGS. 7A and 7B depict another embodiment of the shield 10 designed appropriately for female anatomy. Here, the shield 10 comprises a pad 70 having an opening 72 therethrough that corresponds to the geometry of the drape 12 extending therefrom. In some embodiments, the pad 70 is a shaped as a vertically-oriented elongated oval or “football” shaped member. On either side of the pad 70 is an adhesive tab 71 that is integral with or attached to the sides of the pod 70 and extends therefrom. In some embodiments, the tabs 71 are rounded or semi-circular in shape so as to conform comfortably to the patient's anatomy. Notably, in some embodiments, the pad 70 has no adhesive whereas the tabs 71 do have adhesive such that the tabs will adhere to the external area (vulva) of the patient, while the non-adhesive pad 70 will cover and protect the vaginal opening (introitus). In this configuration, the tabs 71 function as the primary barrier to protect against bacteria entering into the body through the vaginal opening. As in the other embodiments, the catheter 20 is configured to be received through drape 12, with the drape 12 axially aligned with the lumen 23 of the catheter 20. The distal end 21 of the catheter 20 will, in use, extend through the opening 72 of the pad 70 for access to the meatus. In some embodiments, the pad 70 comprises a soft but durable foam material and the tabs 71 comprise a medical-grade adhesive-type bandage material.

These various configurations are intended such that the shield will form, in some embodiments, a complete and secure barrier at and around the target urinary meatus so as to provide a physical barrier preventing the introduction of bacteria to the catheterization site and surrounding areas (e.g. the area at and around the urethra and the spaces or gaps between the urinary meatus and the catheter). Specifically, the physical barrier is effective to prevent bacteria from entering the urinary meatus and to prevent same from traveling through the urinary tract and into the bladder in which the catheter is situated. The use of a shield 10 having a plurality of sections or lobes or other separate ears or features will allow the shield 10 to form fit to a plurality of different anatomies to cover and barrier the exposed urinary meatus while remaining secure for extend periods of time without falling off or otherwise becoming displaced.

In some embodiments, the shield 10 and drape 12 each comprise a non-irritating flexible material that is designed for medium to long-term use without causing damage, irritation, or inflammation to the skin surface on which it is applied. In some embodiments, at least a portion of the shield 10 has an adhesive backing configured to adhere to the skin of the urinary meatus without any harmful interaction or irritation. The shield 10 may include a release layer initially covering the adhesive backing that, when removed, exposes the adhesive backing such that the shield can be applied and adhered to the patient. In some embodiments, to further aid in the protective capabilities of the device, the shield 10 and/or the drape 12 each comprise, contain, and/or are embedded with an antimicrobial, antiseptic, and/or antibacterial material, solution, or coating. Such materials, solutions, or coatings include without limitation silver and silver-containing compounds and chlorhexidine gluconate (CHG) and CHG-containing compounds.

In use, the catheter 20 is first placed into the urinary tract and secured in the bladder in conventional fashion. Next, the shield 10 is brought down to the urinary meatus. The release layer is removed from the shield 10 and the shield 10 is applied and secured at, around, or to the urinary meatus and, optionally, the surrounding skin or tissue. The shield 10 should be applied so as to cover as much of the exposed urinary meatus as possible. A portion of the shield 10 may also come in contact with and adhere to the catheter lumen itself to the extent it is central and proximate to the urinary meatus. Next, optionally the drape 12 is deployed and secured at or around the shield 10, the urinary meatus and/or catheterization site, covering all or a portion thereof. In some embodiments, the drape 12 covers a substantial portion of the surrounding area in a drape or dressing-type arrangement. It is appreciated that both the shield 10 and catheter 12 are designed and configured in a manner not to interfere with the installation, placement, use, or servicing of the catheter or its related components.

While specific embodiments have been described in detail, those with ordinary skill in the art will appreciate that various modifications and alternatives to those details could be developed in light of the overall teachings of the disclosures. Accordingly, the particular arrangements disclosed are meant to be illustrative only and not limiting of the invention, which is to be given the full breadth of the appended claims, and any and all equivalents thereof.

What is claimed is:

1. An antimicrobial, antiseptic, and antibacterial barrier, comprising:
a flexible shield configured to slidingly engage a urinary catheter, said shield comprising one or more sections configured to conform and adhere to the urinary meatus of a target patient;
said shield comprising an antimicrobial, antiseptic, and antibacterial material;
wherein said shield prevents the passage of bacteria, fungi, and contaminants.

2. The barrier of claim 1, further comprising a flexible expandable drape engaged with said shield, said drape comprising an antimicrobial, antiseptic, and antibacterial material; and wherein said drape provides an additional layer to prevent the passage of bacteria, fungi, and contaminants.

3. The barrier of claim 2, wherein said drape comprises an expandable sheath.

4. The barrier of claim 1, wherein at least a portion of said shield has a non-irritating adhesive backing.

5. The barrier of claim 1, wherein said shield comprises a banana-peel configuration having a plurality of elongated said sections.

6. The barrier of claim 1, wherein said shield comprises a flower-petal configuration with four orthogonal said sections.

7. The barrier of claim 1, wherein said shield comprises a circular configuration with four orthogonal said sections.

8. The barrier of claim 1, wherein said shield comprises a condom-like device with a circular cross-section configured to encase the urinary meatus.

9. The barrier of claim 1, wherein said shield comprises an elongated pad, the pad including one or more tabs extending therefrom, wherein said tabs have a non-irritating adhesive backing and said pad is devoid of adhesive.

10. The barrier of claim 9, wherein said pad includes an opening configured to receive a lumen of said catheter.

11. An antimicrobial, antiseptic, and antibacterial barrier system, comprising:
a urinary catheter;
a flexible shield slidingly engaged with said urinary catheter, said shield comprising one or more sections configured to adhere to the urinary meatus of a target patient;
a flexible expandable drape attached to and engaged with said shield, said drape slidingly engaged with said urinary catheter;
wherein said shield and said drape each comprise an antimicrobial, antiseptic, and antibacterial material;
wherein said shield prevents the passage of bacteria, fungi, and contaminants.

12. The barrier of claim 11, wherein said comprises an expandable sheath.

13. The barrier of claim 11, wherein at least a portion of said shield has a non-irritating adhesive backing.

14. A method of preventing the introduction of bacteria to a catheterization site, comprising:
placing a catheter is first placed into a urinary tract;
providing a flexible shield slidingly engaged with said urinary catheter, said shield comprising one or more sections configured to adhere to the urinary meatus of a target patient;
securing said shield to urinary meatus by said one or more sections;
wherein said shield comprising an antimicrobial, antiseptic, and antibacterial material; and
wherein said shield prevents the passage of bacteria, fungi, and contaminants into said catheterization site.

15. The method of claim 14, further comprising the step of deploying a flexible expandable drape engaged with said shield to cover at least a portion of said catheterization site

16. The method of claim 14, wherein said drape comprises an antimicrobial, antiseptic, and antibacterial material.

17. The method of claim 14, wherein said shield comprises a banana-peel configuration having a plurality of elongated said sections.

18. The method of claim 14, wherein said shield comprises a flower-petal configuration with four orthogonal said sections.

19. The method of claim 14, wherein said shield comprises a circular configuration with four orthogonal said sections.

20. The method of claim 14, wherein said shield comprises a condom-like device with a circular cross-section configured to encase the urinary meatus.
